(19) United States
(12) Patent Application Publication
(10) Pub. No.: US 2025/0248584 A1
(43) Pub. Date: Aug. 7, 2025

(54) METHOD AND SYSTEM FOR GENERATING AN AUGMENTED IMAGE OF AT LEAST A PARTIAL SECTION OF A ROW OF TEETH, AND COMPUTER PROGRAM PRODUCT

(71) Applicant: Carl Zeiss Meditec AG, Jena (DE)

(72) Inventors: Max Pfeiffer, Koenigsbronn (DE); Johannes Rangel, Aalen (DE)

(21) Appl. No.: 19/044,328

(22) Filed: Feb. 3, 2025

(30) Foreign Application Priority Data
Feb. 2, 2024 (DE) 10 2024 200 966.0

(51) Int. Cl. A61B 1/00; A61B 1/247; G06T 3/60; G06T 7/00; G06T 7/33; G06T 7/73; G06T 7/80; H04N 13/111; H04N 13/156

(52) U.S. Cl. CPC A61B 1/00009; A61B 1/247; G06T 3/60; G06T 7/0014; G06T 7/344; G06T 7/74; G06T 7/85; H04N 13/111; H04N 13/156; G06T 2207/10012; G06T 2207/10056; G06T 2207/30036; G06T 2207/30204

(57) ABSTRACT

A method is for generating an augmented image of at least a partial section of a row of teeth, wherein a) an imaged mirror image is detected in an image generated by an image capturing device, the mirror image being provided by a mirror element that is arranged in such a way in a capture region of the image capturing device that a mirror image of the at least one partial section of the row of teeth can be imaged by the image capturing device, b) a pose of the mirror element is determined, c) a section corresponding to the partial section of the row of teeth is determined in preoperatively generated information, at least depending on the pose of the mirror element, and, d) the augmented image is generated by virtue of the corresponding section being at least reflected and superimposed on the imaged mirror image.

### CROSS-REFERENCE TO RELATED APPLICATIONS

This application claims priority of German patent application no. 10 2024 200 966.0, filed Feb. 2, 2024, the entire content of which is incorporated herein by reference.

### TECHNICAL FIELD

The disclosure relates to a method and to a system for generating an augmented image of at least a partial section of a row of teeth, and to a computer program product.

### BACKGROUND

So-called preoperative information that serves to diagnose and/or plan the procedure is determined, especially in the case of medical procedures, for example in the field of implantology or for a restorative/aesthetic treatment of a patient. For dental applications, such preoperative information may be generated by what is known as an intraoral scan for example, wherein such a scan detects information about a geometric shape of the teeth. This may then serve to determine a tooth or jaw status, especially in order to document this at various times during treatment. Furthermore, such an intraoral scan may serve to plan or construct an implant or a crown.

Generation of preoperative information via computed tomography-based or magnetic resonance imaging-based methods is also known, wherein, in relation to a tooth in particular, interior structures such as for example, roots or nerves may be detected or imaged as a result. This information may also serve to plan a procedure, for example in order to determine a desired position and orientation of a drill head for drilling purposes.

In addition to anatomical information, the preoperative information may therefore also include further additional information, for example planning information. For example, such planning information may specify which regions of a tooth may be removed for reliable affixing of a crown or where sensitive nerves, which should not be injured during the procedure, extend in the tooth.

During the procedure, the preoperative information may be displayed for example, on a display device, in particular in order to augment intraoperatively generated information. For example, an augmented image depicting information generated both intraoperatively and preoperatively may be provided.

The use of what are known as medical navigation systems is also known, wherein a pose of utilized instruments, inter alia, may be detected using such systems. Depending on this pose, the instrument may be depicted in relation to the preoperative data, for example superimposed on an image generated from preoperative information. Thus the treating physician may for example, obtain information about a relative pose between the instrument and non-visible structures. For example, this may serve to determine a correct drilling angle and a correct drilling depth for a drill. A disadvantage of such navigation systems is that the treating physician must gaze at a monitor during their work if the preoperative data are displayed there, but this does not depict the operating site in real time under certain circumstances. Then, the treating physician must alternate their visual field between the monitor and the examination/operating region. This is disadvantageous, in particular, if the treating physician is performing work using a loupe, which is frequently used, in order to observe the operating region, the loupe generally having a significant weight and not allowing a good visual detection of the examination region in the event of a head tremor.

Surgical microscopes are also known. Such surgical microscopes are used by a user during treatment in order to provide an image, in particular a magnified image, of a treatment region, in particular the situs. So-called stereo surgical microscopes generally include two separate optical channels for beam guidance and may provide the user with a depth impression of the examination region. To this end, the beams guided in the two channels may be captured by the eyes of the user via eyepieces. In an alternative to that or in addition, digital surgical microscopes include two image capturing devices which each capture the beams in one of the optical channels in order to generate an image, wherein the user is then provided with a three-dimensional image via a suitable display device on the basis of the two images, which are also referred to as corresponding images hereinafter. Furthermore, other surgical microscopes that are able to capture optically information, in particular depth information, and able to provide three-dimensional images of a treatment region in particular are also known. To this end, optical detection systems which enable a provision of the depth information on the basis of interferometry, triangulation, time of flight (TOF) or microlens arrays, for example, may also be used in the surgical microscope, in particular in an alternative to a stereoscopy system.

An accurate calibration of the stereo camera system is required to ensure a correct depiction, wherein known calibration methods are used to determine intrinsic and extrinsic camera parameters, which are subsequently used by image processing processes to ensure the correct depiction. Intrinsic camera parameters describe parameters relating to the respective camera/image capturing device itself, for example its distortion. Extrinsic camera parameters describe a relationship, in particular spatial relationship, of the image capturing devices and hence of the camera images to one another. Such intrinsic and extrinsic camera parameters are known to a person skilled in the art.

Known prior art includes US 2023/0320825, which discloses a method and an intraoral scanner for detecting the topography of the surface of a translucent, in particular dental, object.

DE 10 2019 008 510 A1 is also known; it also discloses an intraoral scanner, in particular for the three-dimensional scan of an upper or lower jaw with or without teeth along with jaw components in the context of an implant prosthetic.

DE 10 2016 121 687 A1 is also known; it also discloses an intraoral scanner for the digital dental impression in the dental field and a method for generating a digital dental impression via an intraoral scanner.

EP 3 689 295 A1 is also known; it discloses dental observation equipment, wherein a so-called dental microscope is used.

As a rule, what are known as dental mirrors are used for treatment of a tooth back side or of the molar teeth and optionally also of a chewing surface of a tooth. These can provide a mirror image of a tooth/a row of teeth. Such mirror elements are also used whenever use is made of an image capturing device, in particular a surgical microscope, in order to generate an image, in particular a magnified image, of the oral cavity during a procedure.

### SUMMARY

It is an object of the disclosure to provide a method and a system for generating an augmented image of at least a partial section of a row of teeth and to provide a computer program product, which allow correct augmentation of an image with preoperative information when a mirror element is used.

The above object is achieved via various embodiments of the disclosure.

A method for generating an augmented image of at least a partial section of a row of teeth is proposed. In particular, an augmented image may be an image of the real partial section of the row of teeth, which is augmented with computer assistance, in particular by virtue of for example, at least one virtual object and/or additional information being overlaid or superimposed on the image of the real partial section. The augmented image may be displayed to a user, for example, a treating physician or a surgeon, in particular using a suitable display device such as for example, a monitor or a head-mounted display. If, as still explained in detail hereinafter, the image of the real partial section of the row of teeth is generated using an image capturing device of a surgical microscope, then the augmented image may also be provided to a user in such a way that it is optically detectable for the user through an eyepiece of the surgical microscope. To this end, the preoperative information may be introduced into, for example, reflected into, for example, the beam path leading to the eyepiece. In the case of a surgical microscope having a stereo camera system including two image capturing devices, it is possible that augmented images are generated in each case from the images generated by the two image capturing devices. Hence, an augmented image with depth information, that is, an augmented three-dimensional representation, may thus be provided to a user on an appropriate display device or through an eyepiece as well.

In a first step, an imaged mirror image is detected in an image generated by an image capturing device, the mirror image being provided by a mirror element that is arranged in such a way in a capture region of the image capturing device that a mirror image of the at least one partial section of the row of teeth can be imaged by the image capturing device.

A partial section of a row of teeth may in particular include at least one tooth or a part of one tooth. A tooth may also refer to a denture within the meaning of this disclosure.

Thus, in particular, before the imaged mirror image is detected, the aforementioned mirror element may be arranged in the capture region of the image capturing device, wherein a mirror image of the at least one partial section of the row of teeth can be imaged by the image capturing device. Moreover, the detection may be preceded by a generation of an image by the image capturing device. In particular, the mirror element may be a dental mirror or a part thereof. In particular, the mirror element may include or take the form of a mirror surface that reflects radiation. The image of the mirror surface, generated by an image capturing device, that is, the imaged mirror image, is generated by virtue of capturing this reflected radiation. The mirror image generated/provided by the mirror element is thus perceived by virtue of capturing the reflected radiation. Capture by the image capturing device leads to the generation of an imaged mirror image. However, in addition to the imaged mirror image, the image generated by an image capturing device may also include further regions that do not image the mirror image. In other words, the mirror image may be imaged in a partial region of the image generated by an image capturing device. In the imaged mirror image, the at least one partial section of the row of teeth is imaged. The mirror element may further include a frame section that encloses the mirror surface. The mirror element may also include a handle section so that a user is able to position the mirror element in space. The mirror element is preferably a mirror element with a non-curved mirror surface. By preference, the mirror surface is a round surface. However, the use of polygonal mirror surfaces is also conceivable.

However, a surgical microscope which, in addition or in an alternative to the described image capturing device, uses a further optical capturing system that in particular—but not necessarily—is able to provide depth information is also described. Such optical capturing systems were already described in the introduction above. In particular, this further optical capturing system may differ from a stereo camera system.

A pose of the mirror element, in particular of the mirror surface, is determined in a second step. The pose may include a translational and a rotational component. For example, a position of a reference point of the mirror element, for example of a mirror surface center, and an orientation of the mirror element, for example the orientation of a mirror surface normal, may be determined as the pose of the mirror element. It is self-evident that positions of a plurality of reference points or orientations of a plurality of partial sections of the mirror element may also be determined as pose, especially in the case of a curved mirror surface. Exemplary methods for determining the pose are explained in detail hereinafter. The pose may be determined in a reference coordinate system. For example, the latter may be a reference coordinate system of the image capturing device. The reference coordinate system of the image capturing device may be an image coordinate system of the images generated by the image capturing device. It may also be a reference coordinate system of preoperative information, of a surgical microscope or a coordinate system serving as a reference, which will still be explained in detail hereinafter.

In a third step, a section corresponding to the partial section of the row of teeth is determined in preoperatively generated information, at least depending on the pose of the mirror element. In other words, the portion in the preoperative information including information about the section of the row of teeth reflected by the mirror element is thus determined.

Preoperative information may be generated in the form of image or volume data in particular. As already mentioned at the outset, such information may be generated using CT-based methods, MRI-based methods in particular, but also using other methods, in particular imaging methods such as, for example, ultrasound-based methods. The preoperative information may be assigned a reference coordinate system, whereby the information generated preoperatively may also include spatial information. The pose of the mirror element forms an input variable for determining the corresponding section. In order to perform the third step, it may be necessary to perform a registration between the reference coordinate system of the preoperatively generated information and a reference coordinate system of the image capturing device. This registration may be performed before the third step is performed, in particular before the first step is performed. Registration leads to the determination of a reference, both of the preoperative information and of the image, with respect to a shared reference coordinate system, in particular for the information in the image generated by the image capturing device as well. In particular, this shared reference coordinate system may be the reference coordinate system of the preoperatively generated information, the reference coordinate system of the image capturing device, or else a different reference coordinate system, for example a global coordinate system serving as a reference. Should a surgical microscope be used, the shared reference coordinate system may also be a reference coordinate system of the surgical microscope. This will still be explained in detail hereinafter.

In this context, methods of registration are known to a person skilled in the art. For example, a model-based registration may be performed. In the process, features that correspond to already known features, for example, to geometric features, in particular of the jaw or of the tooth section, in the preoperative information may be detected in the image, wherein the registration may then be determined in known fashion on the basis of these corresponding features. For example, the registration may be determined in the form of a transformation matrix which includes a rotational and/or translational component. An edge-based registration may be a model-based registration, wherein the corresponding features are formed for example by a property of at least one edge, preferably of a plurality of edges, both in the image and in the preoperative information. A topography-based registration may also be implemented, especially if a topography can be determined, for example, using a stereo camera system of a surgical microscope. Thus, topographic information may be determined in the at least one image, wherein, in that case, corresponding features or points or sections are detected both in the preoperatively generated information and in this topographic information, which may subsequently be used to determine the registration. For example, if a stereo camera system, in particular of a surgical microscope, is used to generate the at least one image, then it is possible—if corresponding images are generated by the stereo camera system—to generate a three-dimensional image of at least a partial section of the row of teeth, wherein this three-dimensional image forms or provides the topographic information for registration purposes. However, for registration purposes, it is also possible to use only one of the two corresponding images.

For example, a stereo reconstruction method may be used to generate such a three-dimensional image, wherein the imaged mirror images form input images for this method. Such methods are known to a person skilled in the art. In particular, corresponding picture elements may be determined in the two input images in such methods. For example, such corresponding pixels or pixel sets may be determined using a feature matching method. Corresponding methods and features are known to a person skilled in the art. Exemplary features are what are known as SIFT features, that is, features (for/of) a scale-invariant feature transformation. However, it is self-evident that other methods may also be used for the determination, for example variational methods or AI-based methods. Then, three-dimensional coordinates in a reference coordinate system for the three-dimensional image may be determined for an object point or object section imaged in corresponding picture elements or picture element sets, wherein possible reference coordinate systems were already explained hereinbefore. This may also be referred to as a reconstruction.

The three-dimensional image may be generated on the basis of the pose of the mirror element. For example, a reconstruction method may be performed on the basis of the pose of the mirror element. In particular, at least one method step of the reconstruction method may be performed on the basis of the pose. In particular, the pose may be represented by at least one parameter, wherein the at least one method step is performed on the basis of the parameter, or takes into account the parameter during the implementation. By preference, a stereo triangulation reconstruction method is performed in order to determine the three-dimensional image. Stereo triangulation reconstruction methods are known to a person skilled in the art. In this case, a projection matrix which describes a perspective transformation of three-dimensional object coordinates in a reference coordinate system into two-dimensional image coordinates and which is used during the reconstruction may be determined on the basis of the pose of the mirror element and (known) laws of reflection. In other words, the pose of the mirror element influences the projection matrix of both image capturing devices and hence also the stereo triangulation reconstruction performed on the basis of or dependent on these projection matrices. For example, a so-called homogeneous solution method or a so-called inhomogeneous solution method may be applied for the determination of the three-dimensional coordinates. A rectification method for compensating or eliminating nonlinear distortions in the images, in particular, may be performed before the three-dimensional image is determined.

It may also be advantageous to perform a calibration of the at least one image capturing device. For example, known calibration methods may be used to determine intrinsic and extrinsic parameters of the image capturing device, and these are then used by image processing processes in order to ensure the correct depiction. Intrinsic parameters describe parameters relating to the image capturing device itself, for example its distortion. Extrinsic parameters describe a relationship, in particular a spatial relationship, of a plurality of image capturing devices and hence of the images thereof to one another. Such intrinsic and extrinsic parameters are known to a person skilled in the art. By preference, the aforementioned parameters are determined for all operating states or for predetermined operating states of the image capturing device, wherein an operating state is characterized by the set (adjustable) parameters of the image capturing device (for example, zoom, focal point, capture region). Only intrinsic parameters need to be determined for calibration purposes if only one image capturing device is used. Extrinsic parameters should also be determined for calibration purposes if a stereo camera system with two image capturing devices is used.

The augmented image is generated in a fourth step by virtue of the corresponding section of the preoperatively generated information being reflected and superimposed on the imaged mirror image. Methods known to a person skilled in the art may be used for augmentation purposes. In other words, the preoperatively generated information corresponding to the section reflected by the mirror element is superimposed on the partial region of the image in which the mirror image provided by the mirror element is imaged. The preoperatively generated information is also reflected to this end. The preoperatively generated information may be reflected by the application of a transformation matrix, wherein this transformation matrix is determined on the basis of the pose of the mirror element. In other words, it is thus possible to determine how a specific section in the preoperatively generated information is reflected by the mirror element. A virtually reflected image of the preoperatively generated information, which is subsequently used for augmentation purposes, may be determined to this end, for example, in model-based fashion and in a manner determinable by computer assistance. The virtually reflected image may be an image of the preoperative information reflected by a virtual mirror element, wherein the virtual mirror element is taken into consideration in the model-based determination.

By determining the pose, it is possible in particular to determine the mirror plane in the reference coordinate system and restrict the latter by way of an ascertained variable (by the imaging scale). The law of reflection may be applied to this plane, whereby this restricted area may be projected onto the preoperative information. The resultant intersection between projection and preoperative information may now be reflected appropriately and may be virtually superimposed on the imaged mirror image.

Advantageously, this yields an easy-to-implement and correct augmentation of an image with preoperative information when a mirror element is used. In particular, structures of the partial section that cannot be imaged by the image capturing device may be overlaid on the image of the image capturing device, for example, information about nerve pathways in the interior of a tooth. Additionally, the preoperative information and intraoperative information need no longer be provided on different output devices for a user.

In an alternative to that or in addition, it is also possible that not only preoperatively generated information but also intraoperative information, that is, information recorded during treatment, are used to generate the augmented image. For example, information regarding a partial section of a row of teeth may thus be collected and stored during treatment, and this information may then subsequently be used to generate an augmented image. Thus, in this case, a section corresponding to the partial section of the row of teeth may be determined in the intraoperatively generated information, at least depending on the pose of the mirror element, wherein the augmented image is generated by virtue of the corresponding section being at least reflected and superimposed on the imaged mirror image. The use of intraoperatively generated information is advantageous, in particular, if there are different visualization options that are activated at different times. For example, information about a portion of a row of teeth may thus be obtained in a fluorescence mode, and this information may then be used in a normal vision or white light mode for an augmentation. In particular, this information may be processed and analyzed, in particular classified, prior to further use.

In a further embodiment, the image capturing device is arranged outside of the mouth, in particular outside of an oral cavity in which the partial section of the row of teeth is situated, when the image is generated. The oral cavity may denote a space bounded by the lips at the front, by the hard and the soft palate at the top, by cheeks at the sides, and by the floor of a mouth at the bottom. Within the meaning of this disclosure, the oral cavity may include an oral vestibule. The teeth and a tongue, inter alia, are arranged in the oral cavity.

The oral cavity is connected to an external environment (extraoral space) via what is known as an oral fissure. The image capturing device is arranged in this external environment when the image is generated. Hence, the image capturing device may also be referred to as an extraoral scanner. By preference, a distance between the image capturing device and the oral fissure along an optical axis of the image capturing device is greater than 1 cm, greater than 5 cm or greater than 10 cm when the image is generated. In this context, the distance may be determined between a point of intersection of the optical axis with the oral fissure and a predetermined reference point of the image capturing device, for example, a point of intersection of the optical axis with a termination glass pane of the image capturing device. Hence, the image capturing device may be configured, in particular dimensioned, such that the latter cannot be arranged in the oral cavity for image generation purposes.

Hence, an augmented image for a user can be easily and reliably provided with a multiplicity of image capturing devices which need not necessarily be configured for an arrangement in the oral cavity.

In a further embodiment, the image capturing device is an image capturing device of a surgical microscope. The surgical microscope may serve for the magnified depiction of examination objects or regions, especially in medical applications. Hence, partial regions of the mouth or in the mouth, in particular, may be depicted.

The surgical microscope may include the image capturing device. The latter may be configured to generate a two-dimensional image. In this case, the image may be generated with a predetermined number of pixels and hence resolution. For example, an image sensor of an image capturing device may be a CMOS sensor or a CCD sensor. Self-evidently, other sensor types may also be used.

The surgical microscope may also include a stereo camera system with a first image capturing device and a further image capturing device, wherein the image capturing device for generating the image of the mirror image is one of these image capturing devices. The stereo camera system may be a calibrated stereo camera system.

A surgical microscope with a stereo camera system may include two optically separate beam paths, wherein the first image capturing device is arranged and/or configured such that an image may be generated on the basis of the beams guided in a first beam path. The further image capturing device may be arranged and/or configured such that a further image may be generated on the basis of the beams guided in the further beam path. In particular, the images may be generated at the same time. Furthermore, the images generated by the first and further image capturing devices may be referred to as corresponding images. As explained above, these may serve to generate a three-dimensional image.

Furthermore, the surgical microscope may include at least one optical element for beam guiding and/or beam shaping, the optical element being able to take the form of a lens element in particular, wherein the at least one optical element may for example serve to generate a magnified image. Optical properties of the surgical microscope, for example a magnification, a focal point, a zoom, an exposure time and a capture region size, may be adjustable.

It is possible that the surgical microscope additionally includes at least one eyepiece, through which or into which a user may gaze in order to visually capture the image generated by the surgical microscope. In particular, the user may also capture the examination region through the eyepiece. The surgical microscope may include at least one objective or an objective system, wherein the latter includes the at least one optical element for beam guiding and/or beam shaping. The eyepiece may be or have been optically connected to the objective.

The surgical microscope may be part of a microscopy system, wherein the latter may also include not only the surgical microscope but also a stand for holding the surgical microscope. In this context, the stand may be configured such that it allows a movement of the surgical microscope in space, in particular with at least one degree of freedom, preferably with six degrees of freedom, wherein a degree of freedom may be a translational or a rotational degree of freedom. The degrees of freedom in this context may relate to a reference coordinate system. A vertical axis (z-axis) of this reference coordinate system may be oriented parallel to the gravitational force and counter thereto. A longitudinal axis (x-axis) and a transverse axis (y-axis) of this reference coordinate system may in this context span a plane oriented perpendicular to the vertical axis. Furthermore, the longitudinal axis and the transverse axis may also be oriented orthogonal to one another. Moreover, the stand may include at least one drive device for moving the surgical microscope, for example a servomotor. The stand may also include means for transmitting forces/moments, for example, gear and/or coupling units. Hence, the surgical microscope may be mounted or held in movable fashion. Among other things, this allows a user to modify a pose, that is, a position and/or orientation, of the surgical microscope, for example in order to modify a viewing angle on an examination region or in order to view other examination regions.

The surgical microscope may be a dental surgical microscope in particular, which is configured to generate images in dental applications.

Since surgical microscopes are often used for magnified depiction in the field of dental applications as well, this advantageously yields an improved functionality of these surgical microscopes. An augmented magnification can also be provided for the user even in a simple and reliable manner. In particular, there is no need to switch between a magnified view of intraoperative information, for example, by way of a loupe, and a view of the preoperative information in this case.

Alternatively, the image capturing device may also be a head-wearable image capturing device, for example, by way of an appropriately configured carrier system. It is self-evident that other extraoral image capturing devices may also be used. Other extraoral observation equipment, in particular observation equipment worn on the head or carried on the body or on a body part, with an appropriate image capturing device is also conceivable, in particular also in conjunction with a display device such as for example, a display, which is also arranged outside of the mouth and, in particular, stationary relative to the image capturing device, particularly preferably like glasses, headsets, virtual reality headsets, augmented reality headsets and mixed reality headsets.

In a further embodiment, the superposition is additionally implemented on the basis of at least one optical property of the mirror element. In this context, the at least one optical property may be predetermined. Alternatively, the at least one optical property may also be determined in a further step of the proposed method, in particular in image-based fashion, that is, by evaluating at least one image of the mirror element. In particular, an optical property of the mirror element may be a magnification or reduction property. It is self-evident that other optical properties of the mirror element that affect the mirror image imaging may also be taken into account. For example, the optical property may be represented by the projection matrix explained. This advantageously yields great accuracy of the superposition.

In an alternative to that or cumulatively, the superposition is additionally implemented on the basis of at least one imaging property of the surgical microscope. In particular, an imaging property may be a set magnification (zoom), a set focal point or any other imaging property that affects the imaged mirror image. This imaging property may also be represented by the projection matrix explained. This also advantageously yields a very accurate superposition.

In a further embodiment, a portion of the preoperative information is determined at least depending on the pose of the mirror element, wherein the portion of the preoperative information is assigned to an image of a virtual image capturing device, wherein the at least one corresponding section of the row of teeth, which are reflected in a catadioptric system, is imaged in the image of the virtual image capturing device. Furthermore, the augmented image is generated by virtue of the portion of the preoperative information being superposed on the section of the row of teeth, which is reflected in the catadioptric system.

The catadioptric system denotes a relay optical system which includes at least the mirror element and optical elements of the image capturing device, for example, an objective. If a surgical microscope is used, then the catadioptric system may include optical elements of the objective of the surgical microscope. A beam path through the catadioptric system may be determined on the basis of optical properties of the optical elements in the catadioptric system, which are known in advance or determinable, and on the basis of the pose of the mirror element and known laws of optics.

The virtual image capturing device is a model of an image capturing device that is mathematical or physical and, in particular, can be evaluated with computer assistance. Depending on the model, it is possible to generate a virtual image generated by the virtual image capturing device, in particular by way of a computer-implemented calculation of the picture elements. This virtual image depends, inter alia, on parameters of the (modelled) image capturing device and a pose of the (modelled) image capturing device. Should the mirror surface for example, not be curved and have no magnification properties, then the intrinsic parameters of the virtual image capturing device may be equal to the intrinsic parameters of the modelled image capturing device. In particular, a pose of the virtual image capturing device may be determined on the basis of the pose of the mirror element, in such a way that the virtual image of the virtual image capturing device in this pose images the non-reflected section of the row of teeth, which is reflected by the catadioptric system and hence also reflected by the mirror element. The latter denotes the corresponding section.

In addition to the pose of the mirror element, the generation of such a virtual image also depends on the (further) properties of the catadioptric system, for example, a set zoom of an objective.

Then, the augmented image may be generated by virtue of the portion of the preoperative information being superimposed on the section of the row of teeth that is reflected in the catadioptric system. This may be implemented by virtue of the portion of the preoperative information being reflected, in particular on the basis of the properties of the catadioptric system and hence also on the basis of the pose of the mirror element, and subsequently being superimposed on the imaged mirror image. In other words, the portion of the preoperative information may thus be transformed into the reference coordinate system explained, wherein a transformation depends on properties of the catadioptric system. Advantageously, this yields a superposition that is accurate and easy to implement from a computational point of view.

In a further embodiment, the pose is determined by evaluating at least a property of the imaged mirror image or of the imaged mirror element (or of a section thereof). The at least one property may be determined in image-based fashion, in particular by evaluating the image generated by the image capturing device. In particular, the imaged mirror surface or the imaged mirror element may be recognized in such an image, for example by way of object recognition methods known to a person skilled in the art. For example, object recognition methods may be segmentation methods. Thus, for example, the imaged mirror surface, the imaged frame section or an imaged handle section may be recognized in image-based fashion. For example, it is possible to form a section, for example, a frame section, of the mirror element from material with predetermined optical properties, for example, from matte material, in particular in order to allow a reliable detection of this section in the image. Alternatively, a detection may also be implemented by virtue of a user selecting, for example by way of a suitable input device, an image region in which the mirror image or the section to be detected is imaged.

A property of the imaged mirror image or of the imaged mirror element may be a geometric property of the imaged mirror image, for example, a dimensional property such as a dimensional variable. A dimensional variable may be a width, a height, a diameter or any other dimensional variable. Additionally, a property may be a shape property, for example, a geometric shape such as a circular shape, an ellipsoid shape, a rectangular shape or any other geometric shape. In particular, it is possible to determine a shape factor which represents a relationship between an imaged shape and an actual shape, wherein the pose is determined on the basis of the shape factor.

As explained hereinabove, the pose of the mirror element may influence the imaging by the image capturing device. Hence the pose may also influence how an actual property of the mirror image or mirror element is mapped onto a property of the imaged mirror image or mirror element. If it is possible to describe a relationship between the actual property and the property of the imaged mirror image by way of a transformation matrix that depends on the pose, then the pose can be determined on the basis of the actual property and the property of the imaged mirror image or imaged mirror element. The actual property may be already known, for example, determinable from a model, in particular a CAD model, of the mirror element.

If the mirror element or a portion thereof, in particular the mirror surface, is circular and the imaged mirror image is elliptical, then the pose can be determined on the basis of properties of the ellipse, for example the orientation and lengths of the ellipse axes, and the properties of the circular mirror element known in advance, in such a way that the properties known in advance are transformed into the properties of the imaged mirror image. If polygonal mirror elements, in particular equilateral polygonal mirror elements, are used, then at least a part of the pose may be determined by way of a ratio of the edge lengths in the image and the relative position of the edges to one another in the image.

Additionally, the property may be a pose of the imaged mirror image or of the imaged mirror element or of a portion thereof in the image coordinate system. For example, the position may be determined as the position of a reference point, for example, a geometric center. For example, the orientation of an axis of a reference section may be determined as the orientation. For example, if the mirror element includes a handle section, the latter may be recognized in the image and its position and/or orientation may be determined. For example, the orientation of a longitudinal axis of the handle section may be determined.

If the pose is determined by evaluating at least a property of the imaged mirror image or of the imaged mirror element, then this advantageously yields a simple determination of the pose since the images generated in any case may be evaluated for the purpose of determining the pose.

Alternatively, the pose may be determined on the basis of markers. To this end, the mirror element may include or take the form of at least one marker element for determining the pose of the mirror element. Self-evidently, it is also possible that the mirror element includes or takes the form of a plurality of markers, wherein the pose of the mirror element is determinable on the basis of a relative position of these markers that is known in advance.

The marker element may be an active marker element or preferably a passive marker element. It may be configured to be captured by a capturing device. In particular, the capturing device may be an image capturing device. Thus, the marker element may be an optically capturable marker element in this case. For example, it is conceivable that an optically capturable marker includes a predetermined pattern that allows the pose of the marker and hence of the mirror element to be determined. For example, such optically capturable patterns may take the form of QR codes. Additionally, such optically capturable markers may be reflective marker elements, wherein these for example are configured to be reflective for radiation from a predetermined wavelength range, for example, the infrared wavelength range. The image capturing device for optically capturing the marker element may be the image capturing device for imaging the mirror image or a different image capturing device. If the mirror element includes a plurality of marker elements, then a pose of the mirror element may also be determined on the basis of the relative position of the imaged marker elements in the image. Additionally, the pose of the mirror element may be determined at least in part by a stereoscopic determination of the pose of at least a marker element.

The image capturing device and the marker element may be used to perform in particular what is known as a monoscopic pose determination. In this case, the pose may be determined by evaluating a two-dimensional image, in particular exactly one two-dimensional image of exactly one image capturing device. In particular, an evaluation of intensity values of pixels of the two-dimensional image may be performed in order to determine the position. Such methods for image-based position detection using exactly one image capturing device and/or based on exactly one two-dimensional image are known to a person skilled in the art. Should a stereo camera system be used, the pose may however also be determined by evaluating the corresponding images of the image capturing devices.

In particular, the pose may thus be determined by optical tracking methods, wherein an image capturing device in particular is used to determine the pose. Such optical tracking may be marker-based tracking, in which specific visually or optically capturable marker elements, for example, QR codes or differently configured optical patterns, are used in order to determine the pose.

Alternatively, especially in the above-described determination of the pose by evaluating at least a property of the imaged mirror image or of the imaged mirror element, it is also possible to apply tracking methods without markers, wherein features are captured and used to determine the pose in these methods.

However, in an alternative to an optically capturable marker element, it is also possible to use a marker element that is capturable by other means for the determination of the pose of the mirror element, for example a marker element that is capturable magnetically, capacitively, inductively or in radio-based fashion. For example, a marker element may be configured as an RFID tag.

It is also possible that the mirror element includes a pose sensor, for example an initial sensor or a GNSS sensor, wherein the pose is determined on the basis of output signals of such a sensor. In such an embodiment, the surgical microscope may include a receiver device for the output signals generated by the pose sensor or may be connected to such a receiver device.

Self-evidently, it is also possible to determine the pose using a hybrid method, wherein such a hybrid method combines at least two of the methods for determining a pose, explained hereinabove.

In the case of a marker-based determination of the pose there advantageously is a very accurate determination of the pose, which in turn leads to a generation of an accurate three-dimensional image.

A marker element may also be identifiable, in particular bijectively. For example, a pattern of an optically identifiable marker may encode an identity of the marker. Hence the marker element or the mirror element may be identifiable by capturing the marker element. The identity, which is thus determinable, may be assigned properties of the mirror element, in particular the optical properties explained hereinabove. This assignment and the identity and the properties may be stored in retrievable or readable fashion, for example, in a memory device. This advantageously results in a simple determination of the optical properties of the mirror element.

For the purpose of the marker-based determination of the pose, at least a marker element is imaged in a further embodiment by the at least one image capturing device or by a further image capturing device. The at least one marker element is arranged on or formed by the mirror element. The pose is then determined on the basis of at least one property of the imaged marker element. This has already been explained above. The further image capturing device may in particular be a tracking camera or an environment camera of a microscopy system which takes a different form to an image capturing device of the surgical microscope for the purpose of magnifying imaging. This tracking or environment camera may serve in particular for marker-based tracking of further instruments. In both cases, this advantageously yields the simplest possible integration of an optical determination of the pose, in particular when using the surgical microscope or a microscopy system with the surgical microscope.

In a further embodiment, a focal position of the image capturing device is set on the basis of the pose of the mirror element. In particular, this allows a focal position to be set on a point of the mirror surface or on a point that is spaced apart from the mirror surface by no more than a predetermined distance. In particular, the predetermined distance may depend on a depth of field of the image capturing device or of a surgical microscope, in particular be smaller than the depth-of-field range. In this case, the depth of field is known in advance or may be determined. Consequently, this allows a high imaging quality to be obtained for the mirror image, which in turn advantageously increases the accuracy of the generated three-dimensional image. In particular, this also allows the imaged mirror image to be detected more easily and more reliably in the image of the image capturing device.

The radiation captured for generating the images of the image capturing device is filtered in a further embodiment. The filtering is polarization filtering in a preferred embodiment. However, it is self-evident that other radiation filters may also be used. Advantageously, this may suppress unwanted reflections off a tooth surface in the image, whereby a better visualization of the augmentation is made possible in turn. In this case, polarization filtering enables a maximum possible or complete suppression of reflections.

In a further embodiment, the filter element is arranged in an illumination beam path and/or in an imaging beam path, in particular of the image capturing device or of a surgical microscope. If a plurality of imaging beam paths are present, a respective filter element may be arranged in each imaging beam path. Both cases yield a good structural integration of a filter element in an image capturing device or the surgical microscope or a microscopy system, which allows the generation of a high-quality augmented image.

In a further alternative to that or cumulatively, a filter element is arranged on the mirror element. For example, the filter element may be arranged on a mirror surface of the mirror element. Advantageously, there is no need for the integration of an additional radiation-filtering filter element in the image capturing device or in a surgical microscope or microscopy system as a result, since the mirror element provides the desired filter properties.

In a further embodiment, the at least one partial section of the row of teeth is illuminated by radiation with predetermined radiation properties. For example, such radiation properties may be (a) predetermined wavelength(s) of the radiation used for illumination purposes, predetermined intensities or, in a preferred embodiment, predetermined polarization properties or further properties of the radiation used for illumination purposes. Advantageously, this can also reduce reflections off the tooth surface, which in turn has an advantageous effect on the quality of the augmented image.

In a further embodiment, a registration is performed in a reference coordinate system of the preoperatively generated information and a reference coordinate system of the image capturing device, on the basis of an image generated by the image capturing device. This was already explained hereinabove and advantageously allows simple integration of the registration in the proposed method. In particular, corresponding features in an image and in the preoperative information may be detected for registration purposes, for example, by way of detection methods known to a person skilled in the art, wherein the registration is subsequently performed on the basis of these detected features.

In a further embodiment, the image is generated with a first magnification factor, wherein the augmented image is generated with a further magnification factor that differs from, in particular is greater than, the first magnification factor. In other words, the registration may be performed on the basis of an image which was generated using a smaller magnification factor than the image with a magnification factor used for determining the augmented image. This advantageously yields an accurate and reliable registration since a larger region may be imaged as a result of the smaller magnification factor, whereby more information is available for the registration. The magnification for generating the images with different magnification factors may be generated optically or digitally. For example, the image with a first magnification factor may be generated with an optically set magnification, wherein the image is generated with a further magnification factor with an optically and digitally set magnification. This in turn improves the quality of the augmentation.

In a further embodiment, the registration is performed on the basis of the mirror image. In particular, corresponding features in the detected mirror image and in the preoperative information may subsequently be detected, wherein the registration is subsequently performed on the basis of these detected features. It may be necessary to reflect the preoperative information to this end. This advantageously allows simple integration of the registration in the proposed method. If only a reflected section is imaged by the image of the image capturing device, then the detection of the mirror element may also be dispensed with for the registration in this procedure.

A system for generating an augmented image of at least a partial section of a row of teeth is also proposed, wherein the system includes at least one image capturing device and at least one evaluation device. In a preferred embodiment, the image capturing device may be configured in such a way that for the purpose of generating the image of the mouth, that capturing device must or at least may be arranged outside of an oral cavity in particular.

The system may include a stereo camera system having a first and a further image capturing device.

The system is configured to perform a method according to one of the embodiments described in this disclosure, in particular the following steps:
a) detecting an imaged mirror image in an image generated by the image capturing device, wherein the mirror image is provided by a mirror element that is arranged in a capture region of the image capturing device and reflects the at least one partial section of the row of teeth,
b) determining a pose of the mirror element,
c) determining a section corresponding to the partial section of the row of teeth in preoperatively generated information, at least depending on the pose of the mirror element,
d) generating the augmented image by virtue of the corresponding section being at least reflected and superimposed on the imaged mirror image.

The evaluation device may take the form of or include a computing device. A computing device in turn may include or take the form of a microcontroller or an integrated circuit. In this case, the evaluation device may carry out at least one of steps a), b), c), d), but preferably carry out all of these steps.

The system may be a constituent part of a surgical microscope or of a microscopy system, wherein the surgical microscope or the microscopy system may include the stereo camera system and the evaluation device. Further, the system may include a capturing device for capturing a marker element. Further, the system may include a filter element for filtering the radiation that serves to generate the images of the stereo camera system. Further, the system may include an illumination device for illuminating the partial section with predetermined radiation properties.

The system advantageously enables the implementation of a method according to one of the embodiments described in this disclosure, together with the advantages that have likewise already been described.

The system includes a mirror element in a further embodiment.

A computer program product having a computer program is also proposed, wherein the computer program includes software means for carrying out one, several or all steps of a method according to one of the embodiments described in this disclosure when the computer program is executed by or in a computer or an automation system.

A mirror element for generating an augmented image of at least a partial section of a row of teeth is also proposed. The mirror element is used to generate an augmented image of at least a partial section of a row of teeth using a system according to one of the embodiments described in this disclosure. According to the disclosure, the mirror element includes or forms at least one marker element for determining the pose of the mirror element. In an alternative to that or cumulatively, the mirror element includes or forms at least one filter element for filtering the reflected radiation. This and corresponding advantages have already been explained hereinabove.

### BRIEF DESCRIPTION OF DRAWINGS

The invention will now be described with reference to the drawings wherein:

FIG. 1 shows a schematic flowchart of a method according to the disclosure;

FIG. 2 shows a schematic flowchart of a method according to the disclosure in a further embodiment;

FIG. 3A shows a schematic block diagram of a system according to the disclosure in a further embodiment;

FIG. 3B shows a schematic block diagram of a system according to the disclosure in a further embodiment;

FIG. 4A shows a mirror element in a first pose;

FIG. 4B shows the mirror element depicted in FIG. 4A in a further pose;

FIG. 4C shows the mirror element depicted in FIG. 4A in a further pose;

FIG. 4D shows the mirror element depicted in FIG. 4A in a further pose;

FIG. 5 shows a schematic illustration of a mirror element according to the disclosure;

FIG. 6 shows a schematic block diagram of a virtual image capturing device; and,

FIG. 7 shows an augmented image.

### DETAILED DESCRIPTION

Identical reference signs hereinafter denote elements having identical or similar technical features.

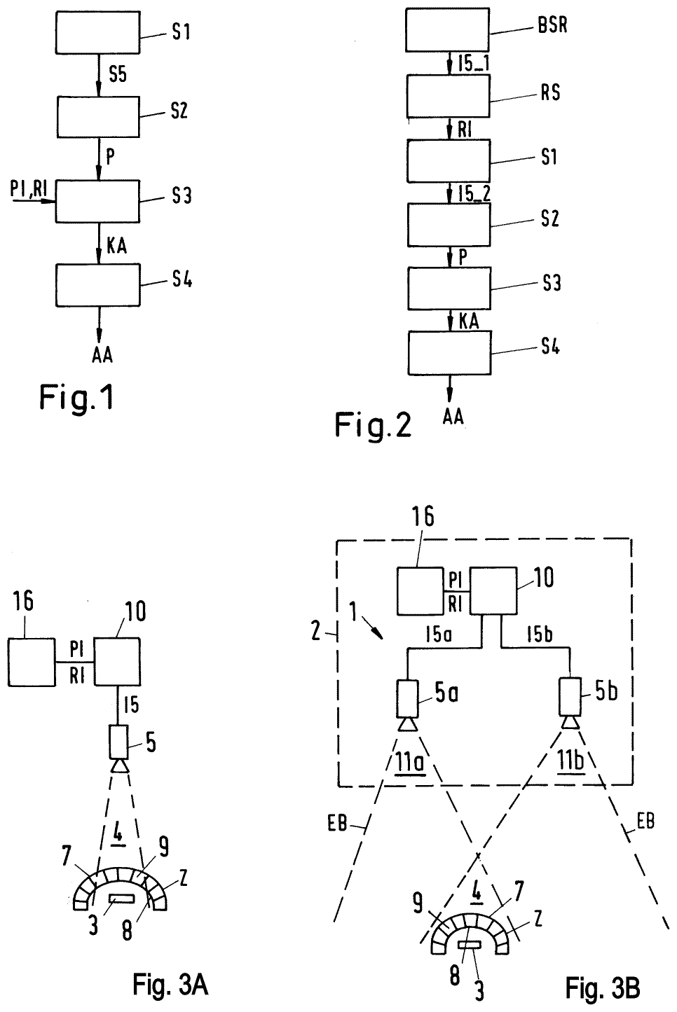

FIG. 1 shows a schematic flowchart of a method according to the disclosure for generating an augmented image AA of at least a partial section of a row of teeth Z (see FIG. 3A). In a first step S1 of the method, an imaged mirror image S5 is detected in an image I5 which was generated by an image capturing device 5 (see for example, FIG. 3A). The mirror image is provided by a mirror element 3 that is arranged in such a way in a capture region 4 of the image capturing device 5 that the mirror image of the at least one partial section of the row of teeth Z can be imaged by the image capturing device 5. This detection in the first step S1 may be performed using a method for object recognition that is known to a person skilled in the art. In addition to a partial region in which the mirror image provided by the mirror element 3 is imaged, the image I5 generated by the image capturing device 5 may include further partial regions in this case, which for example, image other partial sections of the row of teeth Z, in particular those not reflected by the mirror element 3. The imaged mirror image S5 detected thus forms an input variable for determining the augmented image AA.

A pose P of the mirror element 3 is determined in a second step S2. The reference coordinate system may be a coordinate system of the image capturing device 5 serving as a reference or a global coordinate system serving as a reference. It is self-evident that other reference coordinate systems are also conceivable. In addition to the imaged mirror image S5, the pose P forms a further input variable for the determination of the augmented image AA.

A section in preoperatively generated information PI corresponding to the partial section of the row of teeth Z may then be determined in a third step S3, on the basis of the preoperatively generated information PI and registration information RI, which were generated before the third step S3 was performed and before the first step S1 was performed, respectively, on the basis of this pose P of the mirror element 3. The registration information RI allows a transformation of the preoperatively generated information PI and of the image into a common reference coordinate system, which for example may be the coordinate system of the image capturing device 5. The pose P of the mirror element advantageously also allows a transformation into this reference coordinate system.

The augmented image AA is generated in a fourth step S4 by virtue of the corresponding section in the preoperatively generated information being at least reflected and superimposed on the imaged mirror image. This superposition may be implemented using an image fusion method known to a person skilled in the art. In other words, a corresponding section in the preoperatively generated information is superimposed on a partial section of the image in which the mirror image is imaged.

The first step S1 of the method is preceded by a mirror element 3 being arranged in such a way in a capture region 4 of the stereo camera system 1 that a mirror image of at least the partial section of the row of teeth Z can be imaged by the image capturing device 5 (arrangement step). This arrangement is followed by the generation of the image I5 using the image capturing device 5 (image generation step).

FIG. 2 shows a schematic flowchart of a method according to the disclosure in a further embodiment. The implementation of the four steps S1, S2, S3, S4 of the method is preceded in an image generation step BSR by the generation of an image I5_1 with a first magnification factor using an image capturing device 5 (see for example, FIG. 3A). The registration information RI depicted in FIG. 1 is determined in a registration step RS on the basis of the image I5 generated thus. Known methods for image-based registration may be used by a person skilled in the art to this end.

In that case, the first step S1 may be preceded by a modification of the magnification factor for generating the image, wherein the first step S1 is then performed on the basis of an image I5_2 that was generated using a further magnification factor that differs from, in particular is larger than, the first magnification factor.

FIG. 3A shows a schematic block diagram of a system according to the disclosure in a first embodiment, wherein the system includes an image capturing device 5 and an evaluation device 10. FIG. 3A shows that the image capturing device 5 is arranged outside of an oral cavity. A row of teeth Z with a front side 7, a back side 8 and chewing surfaces 9, and a mirror element 3, which is arranged in a capture region 4—denoted by dashed lines—of the image capturing device 5, are also depicted. A mirror element 3, which also reflects beams from the row of teeth Z, in particular from the back side 8 thereof, in particular into an imaging beam path of the image capturing device 5, is also depicted. It is evident from FIG. 3A that the row of teeth Z and the mirror element 3 are arranged in the mouth, with the image capturing device 5 being arranged outside of the mouth. To clarify matters, an oral cavity 17 of the mouth and an oral fissure 18, by way of which the space within the mouth is connected to a space outside of the mouth, are depicted. The image capturing device 5 is arranged in this external space, that is, outside of the mouth or the oral cavity 17. The arrangement outside of the mouth is advantageous in that there is more space for the arrangement and movement of other instruments in the interior of the mouth, and the image capturing device does not as easily come into contact with fluids, bacteria and other substances located in the mouth which may have a contaminating or damaging effect. Hence, appropriate protection or cleaning measures may be dispensed with or avoided. Other extraoral observation equipment, in particular observation equipment worn on the head or carried on the body or on a body part, with an appropriate image capturing device is also conceivable, in particular also in conjunction with displays, which are also arranged in this way, particularly preferably like glasses, headsets, virtual reality headsets, augmented reality headsets and mixed reality headsets.

A memory device 16 for preoperative information PI, which for example, may be retrieved from this memory device 16 by the evaluation device 10, is also depicted. Then, the evaluation device 10 may be used to detect an imaged mirror image, which is provided by the mirror element 3, in an image generated by the image capturing device 5.

A pose P of the mirror element 3 may also be determined via the evaluation device 10 or a further device differing therefrom. Furthermore, steps S3, S4, which are depicted in FIG. 1, may be performed by the evaluation device. The registration information required to this end may likewise be retrieved from the memory device 16, but also from a different memory device.

FIG. 3B shows a schematic block diagram of a system according to the disclosure for generating an augmented image AA of at least a partial section of a row of teeth Z, wherein the system includes a stereo camera system 1 and at least one evaluation device 10. The system is configured to perform at least steps S1, S2, S3, S4 depicted in FIG. 1 and FIG. 2. It is self-evident that the system may also be configured to perform steps BSR, RS depicted in FIG. 2. In this case, the steps or at least parts thereof may be performed by the evaluation device 10.

FIG. 3B schematically depicts capture regions EB of the image capturing devices 5a, 5b and optically separated beam paths 11a, 11b of the surgical microscope 2, which, as is conventional in the art, is arranged outside of the mouth when used as intended. Other extraoral observation equipment, in particular observation equipment worn on the head or carried on the body or on a body part, with an appropriate image capturing device is also conceivable, in particular also in conjunction with displays, which are also arranged in this way, particularly preferably like glasses, headsets, virtual reality headsets, augmented reality headsets and mixed reality headsets. An illumination device of the surgical microscope 2 that is able to illuminate the row of teeth Z is not depicted. In this case, the illumination device may generate radiation with predetermined radiation properties, in particular predetermined polarization properties. The radiation reflected by the row of teeth Z reaches the image sensors of the image capturing devices 5a, 5b via the beam paths 11a, 11b, whereby an image I5a, I5b of the row of teeth Z may be generated. This may be evaluated by the evaluation device 10. A mirror element 3, which likewise reflects beams from the row of teeth Z, in particular the back side 8 thereof, is also depicted, wherein this reflected radiation also reaches the image sensors via the beam paths 11a, 11b and is imaged there as mirror image. This image can then be detected by the evaluation device 10 as imaged mirror image.

It is possible that the radiation captured for generating the images I5a, I5b of the stereo camera system 1 is filtered. This may be implemented by filter elements which for example, are arranged in the beam paths 11a, 11b in each case. Additionally, a filter element may be arranged in an illumination beam path of an illumination device (not depicted) of the surgical microscope 1. Additionally, a filter element may be arranged on/at the mirror element 3. In particular, such a filter element may be a polarization filter element. It is also possible that the evaluation device 10 filters the image I5a, I5b generated by the image capturing devices 5a, 5b, for example in order to suppress reflections.

A memory device 16 for preoperative information PI and registration information RI, which for example, may be retrieved from this memory device 16 by the evaluation device 10, in particular when the third step S3 is performed, is also depicted. The augmented image AA may be generated in the fourth step S4 via the evaluation device 10.

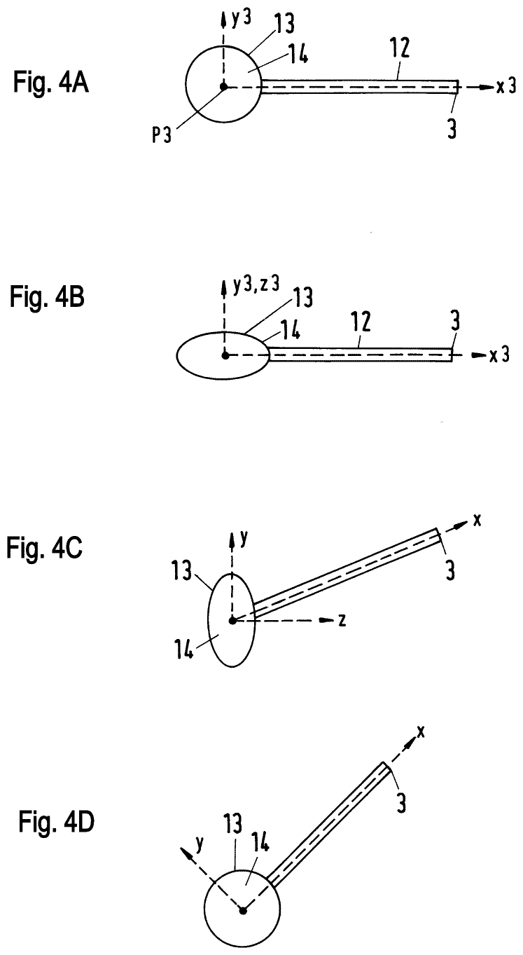

FIG. 4A shows an image of a mirror element 3 in a reference pose. The mirror element 3 includes a handle section 12 and a round mirror section 13, which in turn includes a mirror surface 14. For example, a center of this mirror surface 14 is a reference point P3 of the mirror element 3. A coordinate system that is stationary with respect to the mirror is depicted, it has a longitudinal axis x3 and a transverse axis y3 and a vertical axis z3 (see FIG. 4B).

FIG. 4B shows an image of the mirror element 3 in a pose P which sets in when the mirror element 3 is rotated about the longitudinal axis x3 from the reference position depicted in FIG. 4A. It is evident that the round mirror surface 14 depicted in FIG. 4A is imaged as an ellipse in that case. Depending on an orientation and a length of the major and minor axis of this ellipse, which may for example be detected by way of an object recognition method, it is then possible to determine the rotation angle through which the mirror element 3 was rotated about the longitudinal axis x3, wherein it is then again possible to determine the current pose P of the imaged mirror element 3 depicted in FIG. 4B.

In analogous fashion, FIG. 4C and FIG. 4D show imaged mirror elements 3 which were rotated about the transverse axis y3 (FIG. 4C) or about the vertical axis z3 (FIG. 4D) in comparison with the reference position depicted in FIG. 4A. For example, the corresponding rotation angles may be determined on the basis of an orientation of the longitudinal axis x3 (FIG. 4D) and/or on the basis of the orientation and length of the axes of an elliptical image of the mirror surface 14.

What emerges from FIGS. 4A to 4D is that there may be a shape-based determination of the pose P of the mirror element 3, wherein shape properties of the imaged mirror element 3 may be determined and the pose P may subsequently be determined on the basis of these properties.

It is also evident that a center of the mirror surface 14 is detectable. If the focusing is also directed at a non-reflective edge of the mirror surface 14 in addition to a point reflected on the mirror surface 14, for example, the center, then it is possible to ascertain the distance of the mirror surface 14, in particular the point of reflection, from the row of teeth Z by way of a difference in the focal positions.

In particular, it is possible to determine a difference between the focal position when focusing the stereo camera system 1 or the surgical microscope 2 on a point of the non-reflective edge, for example, a point of the frame section 13, and the focal position when focusing on a point of the object reflected at the mirror surface 14, for example on a point reflected in the center of the mirror surface 14. This difference in the focal positions may represent a distance of the mirror element 13 from the object, in this case a point on the row of teeth Z, wherein the distance may thus be determined on the basis of this difference. Additionally, the distance may also be determined on the basis of the pose P of the mirror element 13. This distance information may be used for scaling within the described stereo reconstruction, in particular in order to match a magnification when reconstructing a reflected section, for example, the back side 8 of the row of teeth Z, to a magnification when reconstructing a non-reflected section, for example, the front side 7 of the row of teeth Z, that is, to carry out scaling.

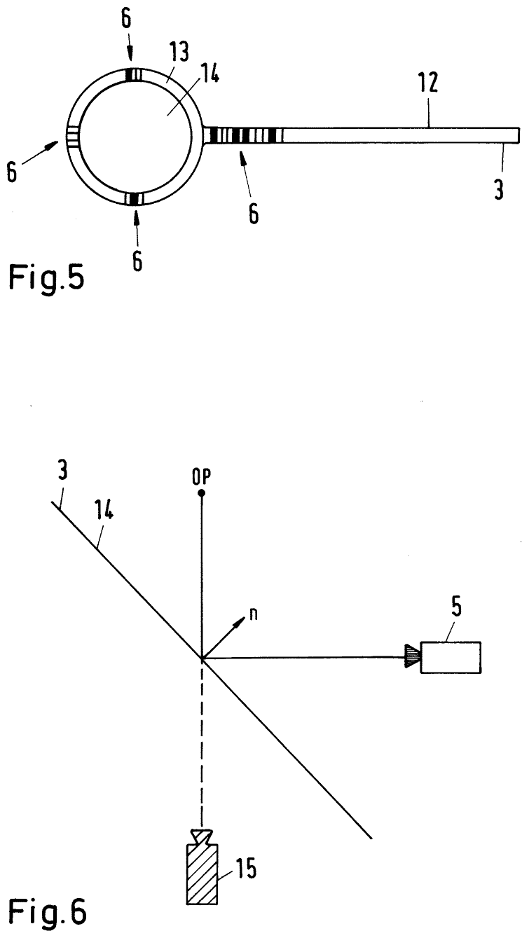

FIG. 5 shows a schematic illustration of a mirror element 3 according to the disclosure. The latter includes or forms a marker element 6, which takes the form of an optically capturable barcode, on a handle section 12. The mirror element 3 also includes further optically capturable marker elements 6, which take the form of a barcode, on a frame section 13 of the mirror surface 14. These may be captured in an image of the mirror element 3, wherein these marker elements in particular enable an identification of the mirror element 3 and a determination of the pose P of the mirror element 3. In particular, each marker element 6 may be detected in the case of the mirror element 3 depicted in FIG. 5, wherein the pose P may then be determined by way of a relative arrangement of the marker elements in the image.

FIG. 6 shows a schematic illustration of an image capturing device 5 (see FIG. 3A) and a mirror element 3, which is arranged in the capture region 4 of the image capturing device 5. An object point OP to be imaged, for example a point on the surface of a row of teeth Z (see FIG. 3A), is also depicted. A normal n of a mirror surface 14 of the mirror element 3 is also depicted.

A virtual image capturing device 15 is also depicted. A (virtual) image of this virtual image capturing device 15 may be determined by evaluating a mathematical or physical model. In particular, the model is determined in such a way that a virtual image of the preoperative information PI is generated, the latter imaging, in non-reflected fashion, a section of the row of teeth Z, that is, in particular the object point OP that corresponds to the section reflected in the catadioptric system, wherein however the properties of the catadioptric system are taken into account. Then, the augmented image AA may be determined on the basis of the virtual image, for example by virtue of the virtual image being reflected and superimposed on the detected mirror image S5.

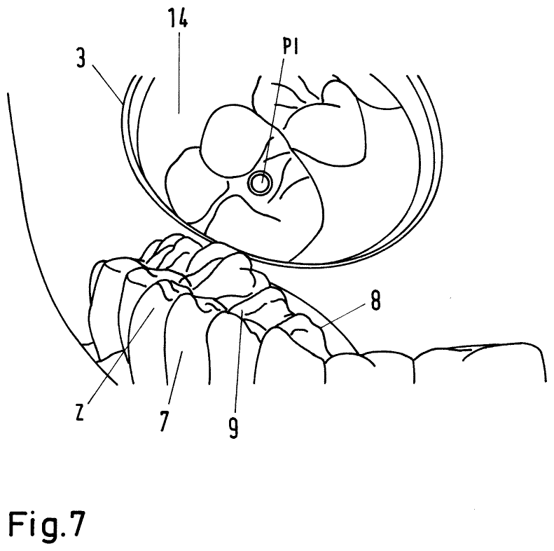

FIG. 7 shows an augmented image AA. Imaged into the augmented image AA are portions of a row of teeth Z. A mirror image S5 of a back side 8 of the row of teeth Z, which is provided by a mirror element 3 with a mirror surface 14, is also depicted. Preoperative information PI in the form of a circle and marking a predetermined section of a tooth is superimposed on this imaged mirror image S5.

In an alternative to the capture by the image capturing device and in a manner analogous thereto, it may also be possible to capture optically information, in particular depth information, using a different capturing system that is used in the surgical microscope and to detect a mirror image, which is provided by a mirror element arranged in the capture region of the surgical microscope and in particular in the capture region of the capturing system, in this optical information, which may be provided in the form of an image in particular, wherein the mirror image inter alia renders optical information of a partial section of the row of teeth capturable for the surgical microscope and in particular for the other capture system. Further, it is possible to determine a section that corresponds to this partial section of the row of teeth in preoperatively generated information, in particular on the basis of the previously determined pose of the mirror element, and to generate an augmented image by virtue of the corresponding section being at least reflected and superimposed on the imaged mirror image.

In an alternative to the use of preoperatively generated information for generating the augmented image or in addition, it is possible that intraoperative information, that is, information recorded or generated during treatment, is used to generate the augmented image. For example, information regarding a partial section of a row of teeth may thus be collected and stored during treatment, and this information may then subsequently be used to generate an augmented image. This is advantageous, in particular, if there are different visualization options that are activated at different times. For example, information, in particular image information, about a portion of a row of teeth may thus be obtained in a fluorescence mode, and this information may then be used in a normal vision or white light mode for an augmentation. In particular, this information may be processed and analyzed, in particular classified, prior to further use.

For example, a point on the surface of a partial section of a row of teeth may thus be contacted or marked with the aid of an instrument, wherein the surface of the partial section is captured by the image capturing device by way of a mirror element. This is one possibility for generating the aforementioned intraoperative information. For example, a marking may then be depicted in the augmented image, in particular on the basis of the pose of the mirror element. For example, the marking may be depicted as a predetermined geometric element. In an alternative to that or in addition, a different sensor system may also be used to capture information about a partial section of a row of teeth. This is a further option to generate the aforementioned intraoperative information, which may then be depicted in the augmented image.

It is understood that the foregoing description is that of the preferred embodiments of the invention and that various changes and modifications may be made thereto without departing from the spirit and scope of the invention as defined in the appended claims.

### LIST OF REFERENCE SIGNS

1 Stereo camera system
2 Surgical microscope
3 Mirror element
4 Capture region
5, 5a, 5b Image capturing device
6 Optically capturable marker element
7 Front side
8 Back side
9 Chewing surface
10 Evaluation device
11a, 11b Beam path
12 Handle section
13 Frame section
14 Mirror surface
15 Virtual image capturing device
16 Memory device
17 Oral cavity
18 Oral fissure
SBV Image generation step
SRV Reconstruction step
I5, I5a, I5b Image
I5_1, I5_2 Image
S5, S5a, S5b Imaged mirror image
P Pose
A1, A2, A Image
OP Object point
EB Capture region
S1 First step
S2 Second step
S3 Third step
S4 Fourth step
AA Augmented image
PI Preoperative information
RI Registration information
KA Corresponding section

1. A method for generating an augmented image of at least a partial section of a row of teeth, the method comprising:
detecting an imaged mirror image in an image generated by an image capturing device, the mirror image being provided by a mirror element that is arranged in such a way in a capture region of the image capturing device that the mirror image of the at least one partial section of the row of teeth is imageable by the image capturing device;
determining a pose of the mirror element;
determining a section corresponding to the partial section of the row of teeth in preoperatively generated information, at least depending on the pose of the mirror element; and,
generating the augmented image by virtue of the corresponding section being at least reflected and superimposed on the imaged mirror image.

2. The method of claim 1, wherein the image capturing device is arranged outside of an oral cavity.

3. The method of claim 1, wherein the image capturing device is an image capturing device of a surgical microscope or an image capturing device that is configured to be worn on a head.

4. The method of claim 1, wherein the superposition is additionally implemented on the basis of at least one of an optical property of the mirror element and an imaging property of the image capturing device.

5. The method of claim 1, wherein an image of a virtual image capturing device is determined at least depending on the pose of the mirror element, the at least one partial section of the row of teeth being imaged without reflection in said image; and, a virtual augmented image is generated by virtue of the corresponding section being superimposed on the non-reflected partial section, with the virtual image being transformed into the augmented image.

6. The method of claim 1, wherein the pose is determined by evaluating at least one property of the imaged mirror image or of the imaged mirror element or in a marker-based fashion.

7. The method of claim 6, wherein, for the marker-based determination of the pose, at least a marker element is imaged by the image capturing device or a further image capturing device; the at least one marker element is arranged on or formed by the mirror element, with the pose being determined depending on at least one property of the imaged marker element.

8. The method of claim 1, wherein a focal position of the image capturing device is set on a basis of the pose of the mirror element.

9. The method of claim 1, wherein radiation captured for generating the images of the image capturing device is filtered.

10. The method of claim 9, wherein the filtering is polarization filtering.

11. The method of claim 9, wherein a filter element is arranged in at least one of an illumination beam path, in an imaging beam path, and on the mirror element.

12. The method of claim 1, wherein the at least one partial section of the row of teeth is illuminated by radiation with predetermined radiation properties.

13. The method of claim 12, wherein the radiation is generated with predetermined polarization properties.

14. The method of claim 1, wherein a registration of a reference coordinate system of the preoperatively generated information and a reference coordinate system of the image capturing device is performed on a basis of the image generated by the image capturing device.

15. The method of claim 14, wherein the image is generated with a first magnification factor, wherein the augmented image is generated with a further magnification factor that differs from the first magnification factor.

16. The method of claim 14, wherein the image is generated with a first magnification factor, wherein the augmented image is generated with a further magnification factor that is greater than the first magnification factor.

17. The method of claim 14, wherein the registration is performed on a basis of the mirror image.

18. A system for generating an augmented image of at least a partial section of a row of teeth, the system comprising:
an image capturing device;
at least one evaluation device;
the system being configured to:
detect an imaged mirror image in an image generated by the image capturing device, wherein the mirror image is provided by a mirror element that is arranged in a capture region of the image capturing device and reflects the at least one partial section of the row of teeth;
determine a pose of the mirror element;
determine a section corresponding to the partial section of the row of teeth in preoperatively generated information, at least depending on the pose of the mirror element; and,
generate the augmented image by virtue of the corresponding section being at least reflected and superimposed on the imaged mirror image.

19. The system of claim 18, wherein the image capturing device is configured such that the image capturing device is arranged outside of an oral cavity for generating the image.

20. The system of claim 18 further comprising the mirror element.

21. A computer program product comprising:
a computer program including program code stored on a non-transitory computer readable medium;
said program code being configured, when executed by a processor of a computer or automation system, to:
detect an imaged mirror image in an image generated by the image capturing device, wherein the mirror image is provided by a mirror element that is arranged in a capture region of the image capturing device and reflects the at least one partial section of the row of teeth;
determine a pose of the mirror element;
determine a section corresponding to the partial section of the row of teeth in preoperatively generated information, at least depending on the pose of the mirror element; and,
generate the augmented image by virtue of the corresponding section being at least reflected and superimposed on the imaged mirror image.

22. A mirror element for generating an augmented image of at least a partial section of a row of teeth by the system of claim 18, wherein the mirror element comprises or takes the form of at least one marker element for determining the pose of at least one of the mirror element and at least one filter element for filtering reflected radiation.
